(19) United States
(12) Patent Application Publication
(10) Pub. No.: US 2021/0398174 A1
(43) Pub. Date: Dec. 23, 2021

(54) SMART HEALTHCARE MANAGEMENT SYSTEM

(71) Applicant: Leonid Shimanov, Jamaica, NY (US)

(72) Inventor: Leonid Shimanov, Jamaica, NY (US)

(21) Appl. No.: 16/905,762

(22) Filed: Jun. 18, 2020

(51) Int. Cl. G06Q 30/02; G16H 80/00; H04W 4/02; H04W 4/30

(52) U.S. Cl. CPC G06Q 30/0283; H04W 4/30; H04W 4/025; G16H 80/00

(57) ABSTRACT

The present invention provides a smart platform for handling a variety of healthcare services offered to patients located at different locations. The smart platform may be configured with an intelligent algorithm to receive a request for a healthcare service from a patient. The algorithm handles the request by negotiating on behalf of the patient and provides cost effective healthcare services to the patient.

### FIELD OF INVENTION

The present invention relates to a method and a system for providing a healthcare service to a patient. More particularly, the present invention provides a smart platform for handling inquiries on healthcare services from the patients and offering lowest price healthcare services.

### BACKGROUND OF THE INVENTION

A number of software platforms present today provide a variety of healthcare services, including dental, nutritional, mental, diagnostic, prenatal, etc. Each healthcare service may be associated with a price, and the platform provides different prices for each healthcare service. Thmay be may be due to the fact that multiple healthcare professionals register with the platform and offer their price, which varies from one healthcare professional to another. Moreover, the software platform adds a subscription fee to each price and offers it to the patients on the platform. The price of each service offered by healthcare service providers to patients may be static and non-negotiable. Moreover, the price offered may be generally on a higher side than the actual cost.

The platform in one form can be considered as a software application installed on electronic devices such as smartphones for providing easy access to the healthcare professional data stored on the platform. Thmay be ease of identifying a healthcare professional offering a particular healthcare service allows more patients to use the software application conveniently and effectively.

However, one of the problems associated with the offerings of healthcare services may be that there are no authenticating means to verify qualifications of an otherwise unknown healthcare service provider, or for assessing the quality of services likely to be rendered from the healthcare service provider.

Presently, there are a number of platforms that provide healthcare services to patients according to their needs. One such example may be described in US patent application 20030195838 assigned to ‘Henley Julian L.’ disclosing a method and a system for implementing an electronic on-line auction of professional services and products via the Internet. Though the invention relates to a technique for reducing transaction costs associated with some professional services and products such as medical services, the invention still lacks a hassle-free process of negotiation. Moreover, the invention doesn't consider the location of the inquiry. Hence, the overall transaction cost doesn't give a clear idea on the actual cost that will be charged, since the overall cost might also include commutation costs for reaching the location of the healthcare service provider.

Another US patent application, US20150317703 assigned to ZocDoc Inc., discloses a system and a method for individualized pricing of healthcare services. The invention overcomes hassle-free negotiation by selecting the lowest price based healthcare service provider based on a combination of a pre-stored pricing and fairness factor associated with each healthcare professional. However, the invention still lacks real-time based negotiation of price as it relies on a weighted average of additional factors, including the fairness factor, etc. Additionally, it doesn't take care of the location of the patient inquiring about the healthcare service.

Yet another US patent application 20180232832 assigned to ‘Jason Szczuka’ discloses a method of determining the likelihood of selection of patient services by a patient by comparing healthcare provider prices from multiple healthcare providers registered with the platform. The invention overcomes hassle by reducing the involvement time. But the invention provides the services to a patient with discounted prices in exchange for promotions of the healthcare provider.

Many of the techniques described above provide different negotiations from either the patient or the platform itself. Since the negotiation from the patient side reduces the chances of arriving at a price point satisfactory to both parties, including the patient and the healthcare service provider, thmay be may be not only detrimental to the platform, but the probability of converting an inquiry of a healthcare service into a deal suffers a lot. Additionally, the patient cannot rely simply on the platform that doesn't offer any negotiation on the price quoted by the patient. In other words, a flat price offered by the platform reduces the chances of converting the query into the deal. Such loses the confidence of the patient to re-visit the platform again, and data traffic may be reduced to a considerable extent.

Hence, there may be a need for an invention that provides a smart platform capable of performing negotiation on behalf of a patient. The platform shall consider the location of inquiry and shall provide the healthcare service at the same location. Moreover, the smart platform shall negotiate with one or more healthcare professionals to select a healthcare professional offering the lowest cost of the healthcare service without exploiting the patient.

It may be apparent now that numerous methods and systems are developed in the prior art that are adequate for various purposes. Furthermore, even though these inventions may be suitable for the specific purposes to which they address, accordingly, they would not be suitable for the purposes of the present invention as heretofore described. Thus there may be a need for a smart platform for negotiating on behalf of the patient and providing the lowest cost healthcare service to the patient at a nearby location.

### SUMMARY OF THE INVENTION

An objective of the present invention may be to provide a platform for facilitating a patient with a healthcare service. The platform may be a smart platform accessible from an electronic device managed by the patient. The electronic device may receive a request for a healthcare service from the patient, along with a price/cost the patient can pay to avail of the healthcare service. A server integrated with the platform and wirelessly in communication with the electronic device receives the request and locates the electronic device. Once the location may be identified, the platform retrieves profiles of healthcare professionals from the same location offering the healthcare service inquired by the patient. Thereafter, the platform negotiates between the healthcare professionals to select a healthcare professional matching the price offered by the patient. The patient may be provided with details of the healthcare professional, including name, address, phone number, etc., for resolving the request. The details are provided on the electronic device handled by the patient. The electronic device may be a Smartphone, a PDA, a desktop computer, etc.

An alternative objective of the present invention may be to resolve the problem of dental care for thousands of patients who are quoted with a fee that they cannot afford. The present invention provides them with a dental treatment at the lowest price possible.

Another objective of the present invention may be to provide a healthcare service such as dental care at a location near the patient with the lowest price possible.

Yet another objective of the present invention may be to provide a smart platform that negotiates on behalf of the patient to choose the lowest price based healthcare professional.

Yet another objective of the present invention may be to provide an intelligent algorithm configured on the smart platform for negotiating on behalf of the patient on a real-time basis.

Yet another objective of the present invention may be to provide an intelligent algorithm configured on the smart platform for directing an inquiry to an administrator for negotiating the price with the healthcare professionals on behalf of the patient.

Yet another objective of the present invention may be to provide a negotiation technique provided by the smart platform to select a healthcare professional by an application representative or a mediator offered by the smart platform for negotiating the cost of the healthcare service between healthcare professionals over the phone call.

Other objectives and aspects of the invention will become apparent from the following detailed description, taken in conjunction with the accompanying drawings, which illustrate, by way of example, the features in accordance with embodiments of the invention.

Embodiments of the present invention may employ any or all of the exemplary aspects above. Those skilled in the art will further appreciate the above-noted features and advantages of the invention together with other important aspects thereof upon reading the detailed description that follows in conjunction with the drawings, which illustrate, by way of example, the features in accordance with embodiments of the invention. The summary may be not intended to limit the scope of the invention, which may be defined solely by the claims attached hereto.

### BRIEF DESCRIPTION OF DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 Illustrate a smart healthcare management system for providing healthcare services to a patient;

FIG. 2 Illustrate an electronic device and a server within the smart healthcare management system;

FIG. 3A Illustrate a negotiation system within the server of the smart healthcare management system where a price may be suggested by the patient;

FIG. 3B Illustrate a negotiation system within the server of the smart healthcare management system where a price may be not suggested by the patient;

FIG. 4 Illustrate a Tele-calling system integrated with the server of the smart healthcare management system;

FIG. 5 Illustrate the server of the smart healthcare management system wirelessly interacting with multiple healthcare professionals;

FIG. 6A Illustrate a method for providing a healthcare service through the smart healthcare management system on a price quoted by the patient; and

FIG. 6B Illustrate a method for providing a healthcare service through the smart healthcare management system when no price may be quoted by the patient.

### DETAILED DESCRIPTION OF DRAWINGS

An objective of the present invention may be to provide a platform for facilitating a patient with a healthcare service. The platform may be specifically a smart platform that may be accessible from an electronic device handled by the patient. The electronic device may be either a Smartphone, a PDA, a desktop computer, etc. The electronic device may receive a request for a healthcare service from the patient, along with a price the patient can pay to avail of the healthcare service.

The platform identifies the location of the patient and provides the lowest price on the healthcare service at the same location. Moreover, the smart platform negotiates between multiple healthcare professionals to select the healthcare professional offering the lowest cost of the healthcare service without consuming the patient's time and effort.

Alternatively, the invention discloses an intelligent platform for providing on-line negotiation on behalf of the patient. A healthcare professional enables a service providing platform to schedule facility utilization and automatically compute an offered price on the basis of the negotiation performed. The negotiation may be done with respect to the price range provided by the patient. A price “nearest” a patient price is no more than 10% over the quoted patient asking price or some predefined value set by the patient. If P wishes to purchase a service within X miles from P, then location Y will be near P if location Y is no more than 2× (default delta value) away from P when there are no services available within X miles of P. Alternatively, the delta value X may be some other predefined system value or a patient input value. For example, if P wishes to purchase a service within 50 miles of P, a location 100 miles from P is near P when there are no services within 50 miles of P.

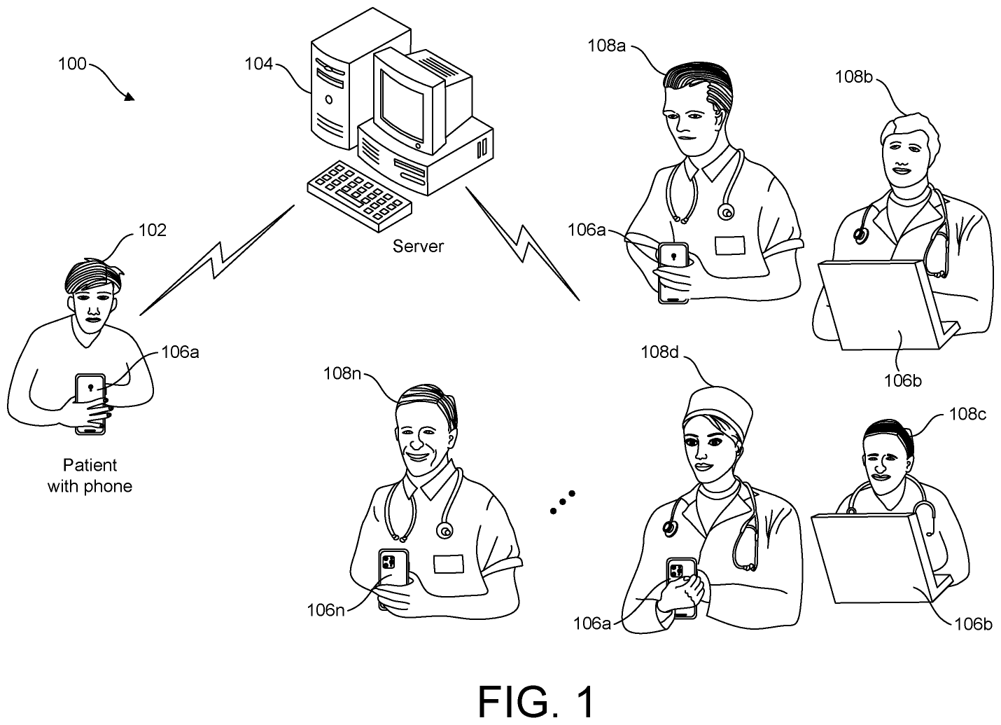

FIG. 1 Illustrate a smart healthcare management system 100 for providing healthcare services to a patient 102. The smart healthcare management system 100 may include a centralized server 104. The patient 102 uses an electronic device 106 for accessing the platform offered by the smart healthcare management system 100 The server 104 may be cloud-based and may provide a marketplace to the patient 102 to choose from a variety of healthcare services at a lowest price. The server 104 may be configured with the smart platform for communicating with the patient 102 and a number of healthcare professionals 108a-108n. The server 104 may include a database that stores all relevant information pertaining to the healthcare professional 108a-108n. More specifically, the profile store expertise offered by each healthcare professional 108a-108n, along with their location and pricing for each healthcare service.

According to one embodiment, the smart healthcare management system 100 works intelligently by including the patient 102 as a payer, the server 104 with relevant information of all healthcare professionals 108a-108n in the database integrated with the server 104. The healthcare professional 108a-108n may be either a dentist, a doctor, a physiotherapist, a psychologist, a psychiatrist, or a pharmacist.

The information stored in the database of the platform may be integrated in a computing device 106a-e. The information stored in the database may be a complete profile of the healthcare professional 108a-108n. The profile may include name, service offered, location, and cost associated with the service. The computing device 106 may be either of a phone 106a, a laptop 106b, a tablet, a desktop computer, or the like. The computing device 106a-b may be wirelessly communicable with the server 104. The wireless communication may be either 3G, 4G, 5G, LTE, or any advanced wireless communication technology to establish communication of the patient 102 with the server 104 placed remotely. The wireless communication technology can also be short-range technology, including Bluetooth, Infrared, or the like.

The platform integrated with the server 104 uses an intelligent algorithm for processing the request received from the patient 102. The request may include a price range and a healthcare service required. In one embodiment, the price provided by the patient 102 may be a flat price. In another embodiment, the patient 102 can define a range of prices within which the patient 102 may be willing to pay for the healthcare service. The platform provides the patient 102 to select from a variety of healthcare services offered, along with a price range defining capability, so that the patient 102 can define these parameters easily. The request may be placed on the interface displayed on the computing device 106a (phone) of the patient 102. In one embodiment, the intelligent algorithm may be a software application that the patient 102 can install on the computing device 106a-e. The request may be forward to the central server 104 for processing.

The server 104 receives the request. The server 104 may include a receiver, a GPS locator, a controller, and a negotiation module. Specifically, the receiver of the server 104 receives the request and sends it to the GPS locator. The GPS locator identifies the location of the computing device 106a-e associated with the patient 102. Once the location may be identified, the GPS locator sends the location of the patient 102 to the controller. The controller integrated with the database identifies the healthcare professional 108a-108n within or near the location and offers the healthcare services inquired.

Based on the selection of the location, the controller retrieves multiple profiles of the healthcare professional 108a-108n within or nearby the location. The retrieved profiles of the healthcare professional 108a-108n are compared with the request made by the patient 102. Primarily, the controller may include two distinctive comparators for comparing healthcare services. The comparator may include a service comparator for comparing the healthcare services offered by the healthcare professional 108a-108n and a price comparator for comparing the prices of the healthcare services with the price range requested by the patient 102. Alternatively, the controller may include a single comparator, which may be a service comparator for comparing the healthcare services within the location and identifying the healthcare professional 108a-108n offering lowest price based healthcare services.

The smart healthcare management system 100 with the intelligent algorithm allows the platform to utilize one or more negotiation techniques for reducing the cost of the healthcare service. The negotiation techniques can include one or more of Anchoring, Whack Back, Sticker Shock, etc.

The healthcare professional 108a-108n can reduce their prices according to the negotiation done with the negotiator module within the server 104. The negotiation may be done in view of the price range requested by the patient 102. The negotiation module within the server 104 primarily performs the negotiation by applying various rules to reach a price that may be closer to the price requested. In an alternative embodiment, the negotiation may be performed by a mediator of the third party, which can be a professional having knowledge about the field, such as a representative over a phone, including a tele-caller.

The short-listed profiles of the healthcare professional 108a-108n after negotiation may be shared back to the computing device 106a-b by the server 104. The short-listed profiles may be displayed on the computing device 106a-b of the patient 102. The patient 102 may select any suitable doctor's profile afterward and resolve the healthcare service inquiry. The server 104, thereafter, closes the request and charges a small amount from the patient 102 for providing hassle-free and lowest healthcare service to the patient 102. In one embodiment, the server 104 may also charge a convenience fee from the healthcare professional 108a-108n as well. The interface provided by the server 104 may also include a payment module that allows transactions between the patient 102 and the healthcare professional 108a-108n in a hassle-free manner. The payment module may accept payments via either of a Credit card, a Debit Card, PayPal, Vemo, etc. Alternatively, the payment can be made in cash once the healthcare service may be availed by the patient 102 from the short-listed healthcare professional.

In a preferred embodiment, the patient 102 and the healthcare professional 108a-108n use the cloud-based technology for accessing features provided by the server 104 and generating a real-time price comparison with the intelligent algorithm. The patient 102 may use a mobile computing device 106a-b to access a cloud-based marketplace through the Internet. The communication between healthcare professionals 108a-108n and the negotiating module with the server 104 may include a secure login procedure to limit any unauthorized access. The healthcare professional 108a-108n can then review the negotiator statistics, prices, and other details to make a decision on the negotiation to be made. In an alternative embodiment, the healthcare professional 108a-108n negotiates with a tele-caller representative managed by the server 104 over the phone.

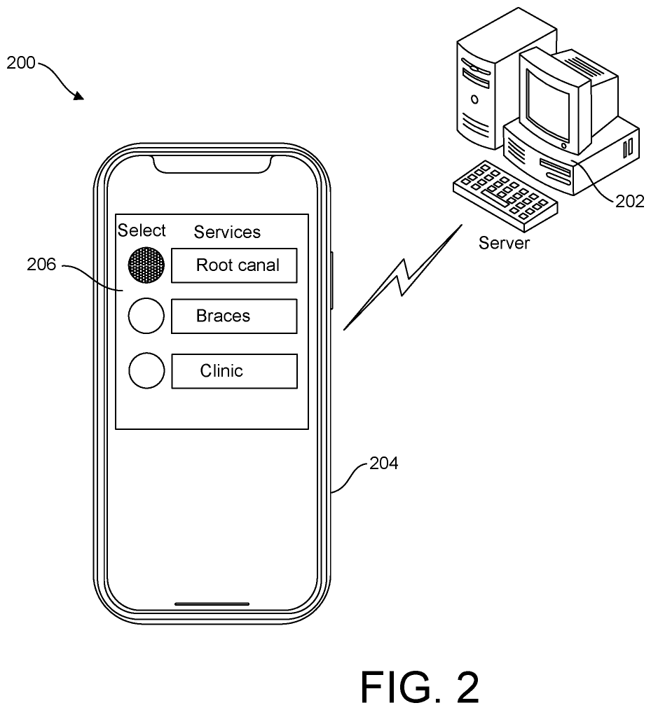

FIG. 2 Illustrate an electronic device 204 and a server 202 within the smart healthcare management system 200. The electronic device 204 may be provided with an interface 206 from the server 202 to place a healthcare service request. The patient 102 using the electronic device 204 can provide text, voice, or any other form of typing input to the interface 206. Specifically, the patient 102 may provide secure login credentials on the interface 206 of the platform displayed on the electronic device 204. Thereafter, the patient 102 can choose or select the healthcare service of interest. In one embodiment, the patient 102 may provide touch-based input to the electronic device 204. In another embodiment, the patient 102 can provide voice-based input recognized by the interface 206. The electronic device 204 can be either of a phone, a laptop, a desktop computer, or the like.

Primarily, the healthcare service request can be made via a text given on the information column of the interface 206. By checking a suitable box on the platform with the healthcare service of interest, the interface 206 inquired on the price the patient 102 may be willing to pay. Alternatively, the healthcare service request may be made by making a call to the mediator provided by the platform. The healthcare service request may include information on the healthcare service required, along with price quoted by the patient 102. The request may be communicated (wired/wirelessly) to the server 202. At the server 202 side, the request may be received and forwarded to a GPS locator for locating the electronic device 204. Once the location is identified, the server 202 locates healthcare professionals offering services at the location of the electronic device 204. The server 202 negotiates on behalf of the patient 102 to choose and provide the lowest healthcare professional offering desired services within the nearby location of patient 102.

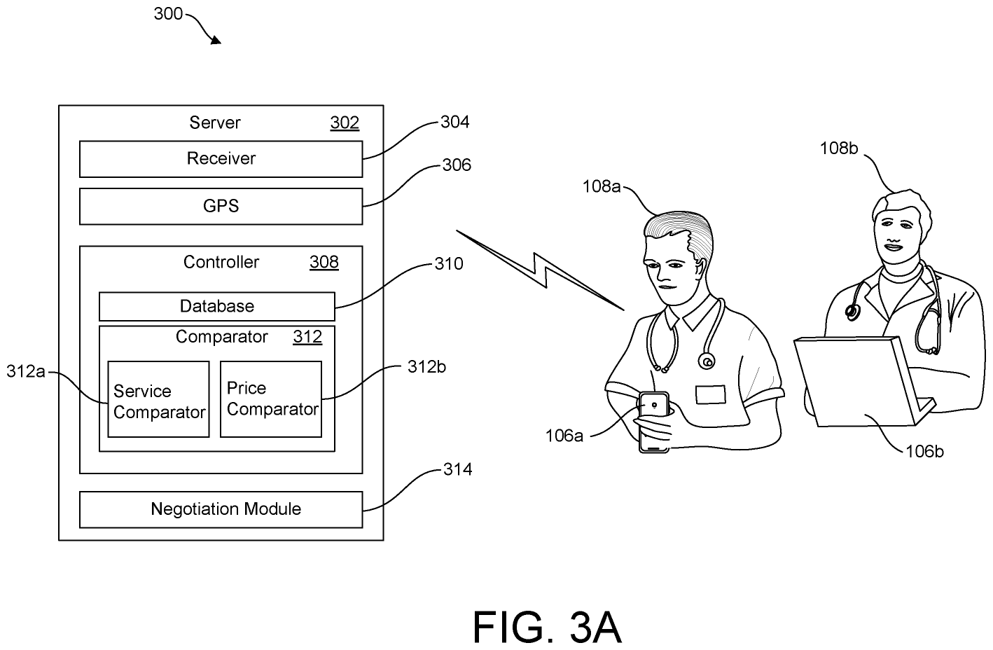

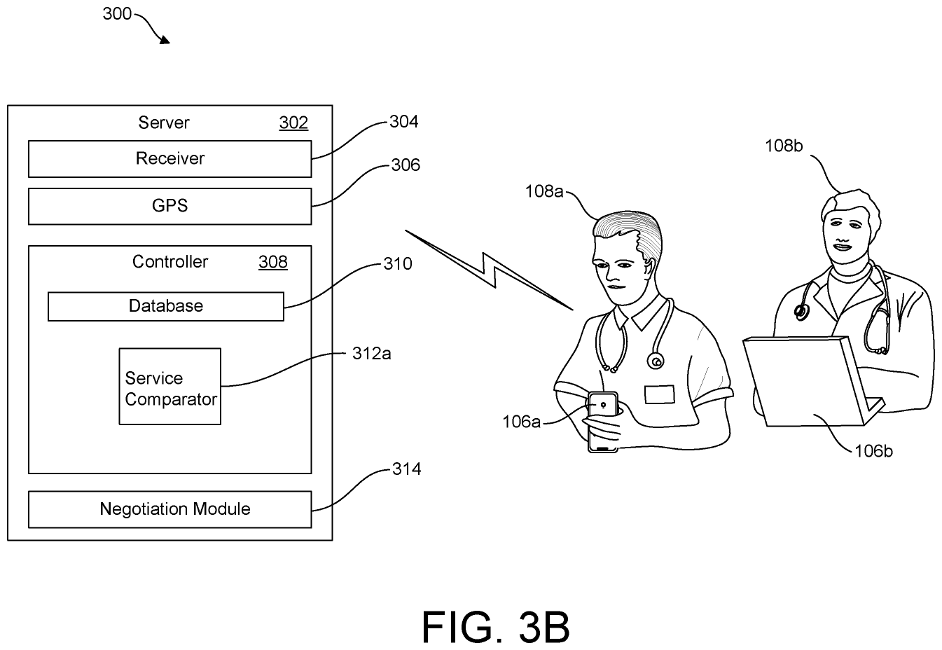

FIG. 3A Illustrate a system architecture 300 that establishes communication between the server 302 of the smart healthcare management system 100. The server 302 may include a receiver 304, GPS locator 306, a controller 308, and a negotiation module 314. The controller 308 further may include a database 310 that stores the profile of the healthcare professionals 108a-108n. The controller 308 also may include a comparator 312. The comparator 312 may include two comparators, including a price comparator 312a and a service comparator 312b. Each of the comparators 312 contributes towards a short listing of healthcare professionals 108a-108n. The negotiation module 314 may be capable of negotiating with one or more healthcare professionals 108a-108n based on a selection of profiles by the price comparator 312a and the service comparator 312b. The comparator 312 performs matching of the inquired healthcare service with the stored profiles and provides one or more healthcare professionals 108a-108n satisfying the comparison. In one embodiment, the comparator 312 may be able to access the most pertinent data relating to the patient's 102 location. The short-listed healthcare professionals 108a-108n are then contacted, and a reasonable negotiation may be performed. The negotiation may be based on rules applied to the smart algorithm. The negotiation may be performed using criteria such as the price offered by the patient 102 and associated location. In an alternative embodiment, a weighted average of the factors, including price and location, may be performed to generate a numerical value associated with each short-listed healthcare professional 108a-108n to make a decision. The weighted average may be performed by the negotiation module 314, so that the patient 102 gets hassle-free access to most appropriate healthcare professionals 108a-108n handling the healthcare service inquiry. In another embodiment, only the price offered by the patient 102 may be the selection criteria for most appropriate healthcare professionals 108a-108n.

The negotiation module 314 may negotiate with one or more healthcare professionals 108a-108n for providing the one or more services matching the request at a price offered by the patient 102. The price negotiated may be either less than the price asked by the patient 102 or may be closest to the price the patient 102 suggested. The profiles of the healthcare professional 108a-108n may be displayed on the electronic device 304 based on the negotiation. So, the patient 102 can choose one of the healthcare professionals 108a-108n that most suitably matches the requirements. Alternatively, each healthcare professional 108a-108n can also be provided with ratings to aid in the selection of healthcare professional 108a-108n by the patient 102 in certain circumstances. Some other factors may include the experience of healthcare professionals 108a-108n etc.

FIG. 3B Illustrate an alternate system architecture 300 that establishes communication between the server 302 of the smart healthcare management system 100. The system architecture 300 may only accept inquiry on a particular healthcare service that the patient 102 wishes to avail through the platform on the server 302. The request may be received by the server 302 wirelessly through the receiver 304. The receiver 304 supports any of the known in the art wireless interface standards such as IEEE 802.11a, 802.11b, etc. The receiver 304 forwards the request to the GPS 306, which in turn identifies the location of the electronic device 104 that initiated the request. Once the location is identified and confirmed, the controller 308 may initiate a search on the database 310 to select healthcare professionals 108a-108n within the location. In one embodiment, if the search doesn't yield a suitable healthcare professional 108a-108n within the location, the controller 308 may expand the search area by selecting a nearby location to the identified location, to identify a suitable healthcare professional 108a-108n. Once the search is completed, the short-listed healthcare professional 108a-108n may be contacted, and a reasonable negotiation may be performed by the negotiation module 314 on behalf of the patient 102. The negotiation may be done based on rules applied in the algorithm consistent with the price entered by the patient 102. The negotiation techniques can include one or more of Anchoring, Whack Back, Sticker Shock, etc. The negotiation techniques may be integrated with the smart algorithm managed by the controller 308.

The negotiation module 314 may not perform the negotiation according to the patient 102 input, as the reference cost is generally not provided by the patient 102. The negotiation with the healthcare professional 108a-108n may be performed based on the market price of that particular service. The negotiation module 314 may decrease the market rate by some predefined percentage and start the negotiation with the doctors. So, the price negotiated may be either less than the price or close to the price of the service. The profiles of the healthcare professional 108a-108n are displayed on the electronic device 104 after the negotiation is completed. Thereafter, the patient 102 can choose the lowest cost healthcare professional 108a-108n. Alternatively, each healthcare professional 108a-108n can also be provided with ratings to aid in the selection of healthcare professional 108a-108n by the patient 102 in certain circumstances. Some other factors may include the experience of healthcare professionals 108a-108n etc.

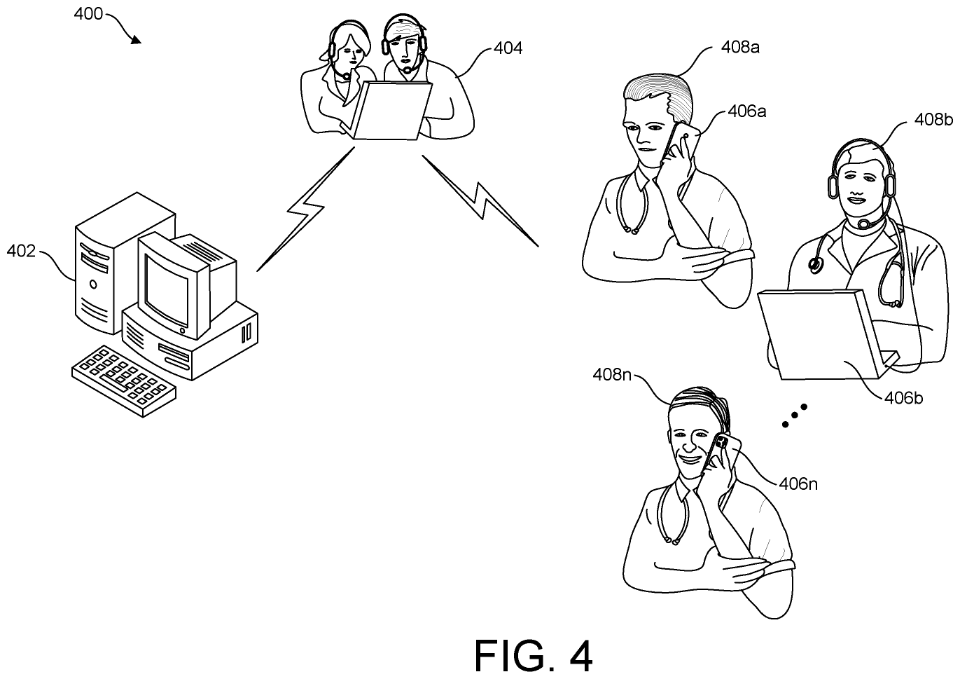

FIG. 4 Illustrates system architecture 400 with a server 402 connected with a Tele-calling system 404 of the smart healthcare management system 100. The system 400 may allow the request of the healthcare service from the patient 102 to be directly routed to the Tele-calling system 404 from the server 402. The request processing arrangement may be quite similar to the arrangements previously described except, for this embodiment, the negotiation module 314 may be absent. Instead of using a negotiation module 314, the negotiations with respect to the price and the service may be performed by the Tele-calling system 404 with executives by calling the short-listed healthcare professional's computing device 406a-406n from the server 402. So, a tele-caller executive or a mediator from the Tele-calling system 404 performs the negotiation with the healthcare professional 408a-408n to select one healthcare professional 408a-408n that offers the lowest price on the service requested.

In one embodiment, the Tele-calling system 404, with executive, may decrease the market rate by some percentage and start the negotiation with the healthcare professional 408a-408n. So, the price negotiated may be either less than the price asked by the patient 102 or may be close to the price the patient 102 requested. The profiles of selected healthcare professionals 408a-408n are displayed on the electronic device 406a-406n based on the negotiation.

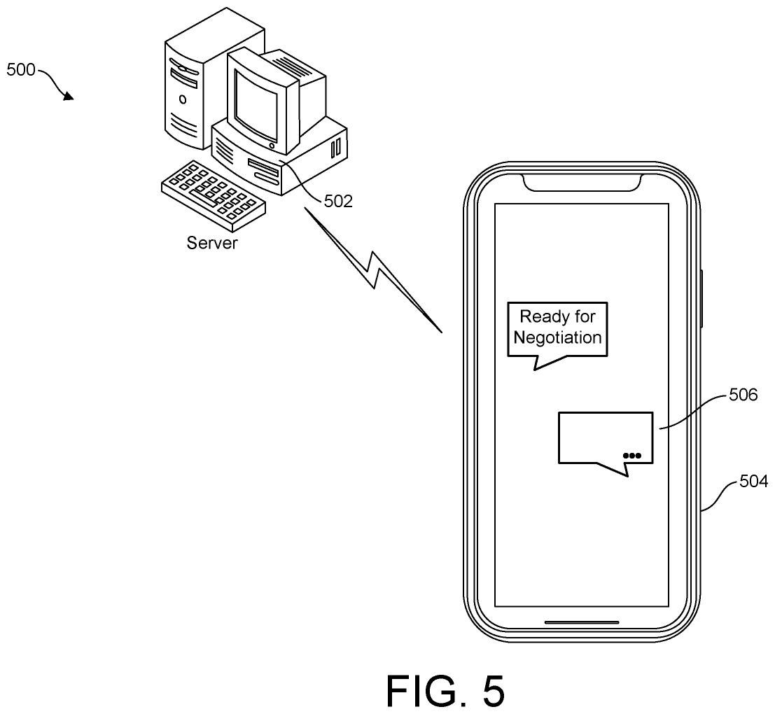

FIG. 5 Illustrates a system architecture 500 with a server 502 connected with an electronic device 504 managed by the healthcare professional 408a-408n of the smart healthcare management system 100. The short-listed profiles of the healthcare professional 408a-408n may be negotiated through an interface 506 provided by the server 502. The healthcare professional 408a-408n interacts with the server 502 through an interface 506. The interface may provide a real-time chat messenger to healthcare professional 406a-406n for negotiating on the costs. In one embodiment, the interface 506 may also provide a facility to call through the server 502 directly to the patient 102 to interact once the negotiations are done. Additionally, the interface 506 may also provide an option to opt-out at any time during the negotiation if the healthcare professional 408a-408n is not further interested in negotiating on the price.

In another embodiment, the interface 506 may provide the healthcare professional 408a-408n a direct dial-in feature allowing him/her to speak with a tele-executive handled by the server 502 for negotiation vocally. The tele-executive may directly interact with the healthcare professional 408a-408n to close the inquiry in a possible way. Specifically, the tele-executive may negotiate on behalf of the patient 102 to reach a price close to the cost offered by the patient 102. Alternatively, if the tele-executive doesn't find the negotiation on a positive side, the tele-executive can look for another healthcare professional 406a-406n from the short-listed healthcare professionals 408a-408n. The patient may predefine what price is “close” to the desired price. A default “close” price may be no more than 10 percent over the asking price. Alternatively, the patient 102 may set the “close” value.

The interface 506 typically contains multiple interactive features, including secured login, notification triggers, sound triggers, chat messenger, a direct dial-in calling feature, etc. Moreover, the healthcare professionals 408a-408n who want to provide healthcare service can directly go to an App Store, iCloud, and register themselves. Thereafter, the healthcare professional 408a-408n can create a profile on the platform offered by the server 502 to market their healthcare services.

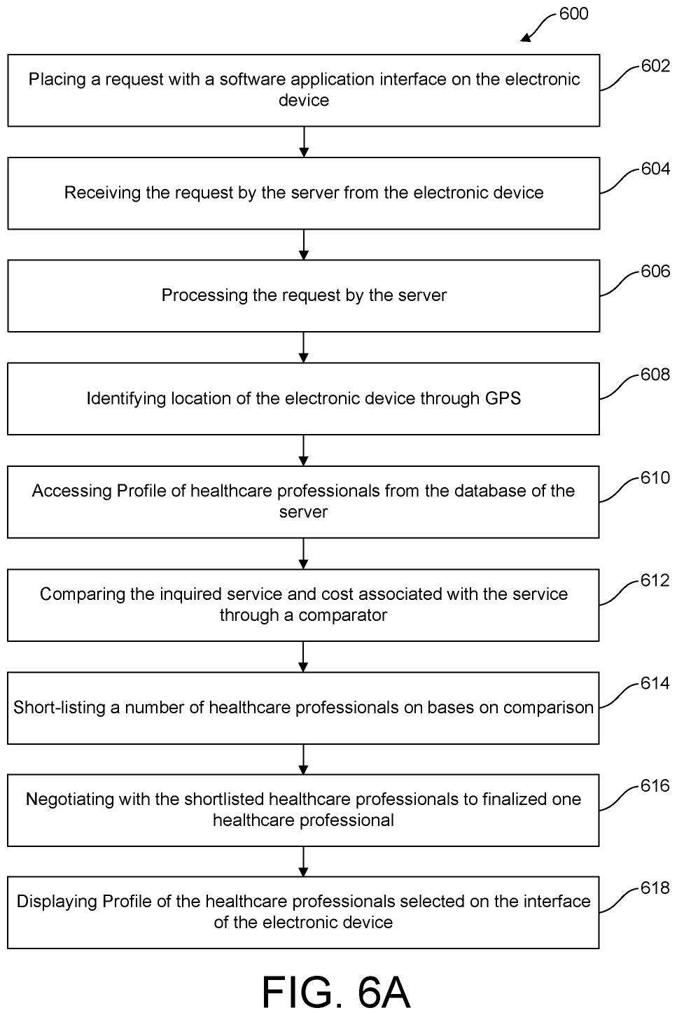

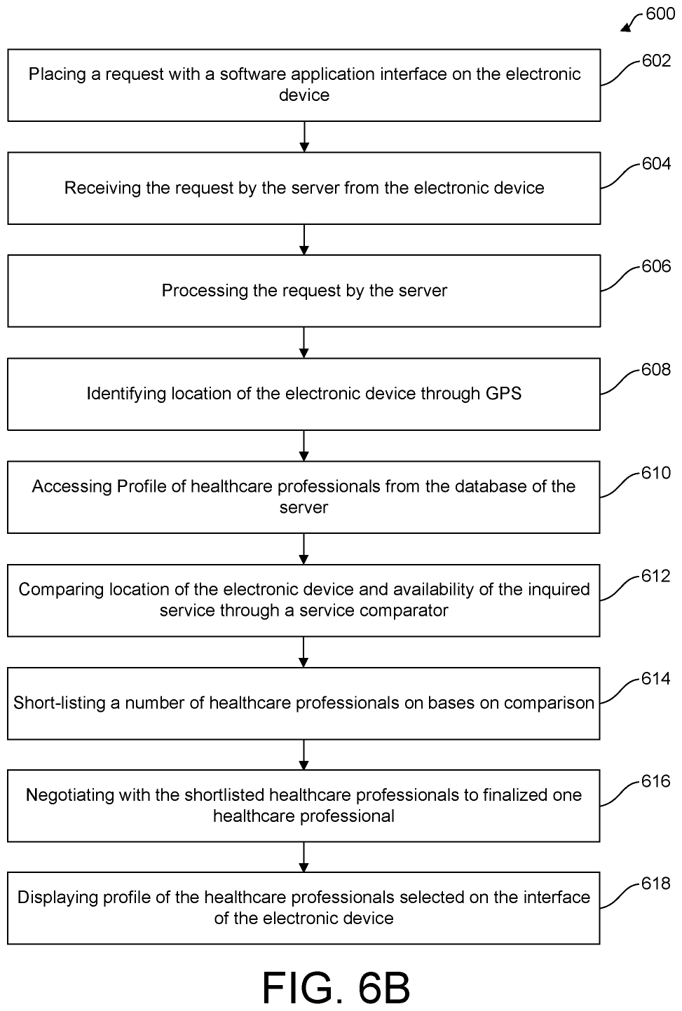

FIG. 6A represents a flow chart disclosing a method of providing healthcare services through the smart healthcare management system 600 based on a price quoted by the patient 102. The method may include step 602 of placing a request with a software application interface on the electronic device 106, for the healthcare service, along with an associated price. Thereafter, step 604 may involve receiving the request by the server 104. The request may include the healthcare service and the associated price. The request may be processed by the server 104 in Step 606 through a software platform. The processing may further involve step 608, which identifies the location of the electronic device 106. Thereafter, step 610 may be executed to provide access to the healthcare professionals from a database managed by the server 104.

Once the healthcare professionals' locations near the location of the electronic device 106 are determined, step 612 compares the inquired healthcare service and associated cost with the one or more services offered by the plurality of healthcare professionals stored in the database. Step 614 may then provide a short-listing of the healthcare professionals based on a comparison in step 612. After the short-listing is done, step 616 may provide further negotiations with the healthcare professionals to select one healthcare professional offering the lowest price on the healthcare service. Lastly, step 618 may display the selected healthcare professional on the electronic device 106 to the patient 102.

FIG. 6B represents a flow chart disclosing an alternate method of providing healthcare services through the smart healthcare management system 600 when the patient 102 does not quote a price associated with the healthcare service. The method may include the step 602 of placing a request with a software application interface on the electronic device 106 for the healthcare service. Thereafter, step 604 may receive the request by the server 104. The request may include the healthcare service the patient 102 wishes to avail. The request may be processed by the server 104 in Step 606 through a software platform. The processing may further involve step 608, which identifies the location of the electronic device 106. Thereafter, step 610 may be executed to provide access to the healthcare professionals from a database managed by the server 104. Once the healthcare professionals are located near the location of the electronic device 106, step 612 compares the inquired healthcare service with one or more services offered by the plurality of healthcare professionals stored in the database. Step 614 may provide a short listing of healthcare professionals based on a comparison in step 612. After the short-listing is done, step 616 may provide further negotiation with the healthcare professionals to select one healthcare professional offering the lowest price on the healthcare service. Lastly, step 618 may involve displaying the selected healthcare professional on the electronic device 106 to the patient 102.

While the various embodiments of the present invention have been described above, it should be understood that they have been presented by way of example only, and not of limitation. Likewise, the figure may depict an example architectural or other configuration for the invention, which may be done to aid in understanding the features and functionality that can be included in the invention. The invention may be not restricted to the illustrated example architectures or configurations, but the desired features can be implemented using a variety of alternative architecture and configurations.

Although the invention may be described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case may be intended or required in instances where such broadening phrases may be absent.

1. A platform for providing a patient with a healthcare service, the platform comprising:
(A) an electronic device for receiving a request from the patient, wherein the request may be based on the healthcare service and a price related to the healthcare service;
(B) a server, wherein the server includes:
1) a receiver for receiving the request;
2) a GPS for locating the electronic device; and
3) a controller for processing the request comprising:
a) a database, wherein the database maintains profile of healthcare professionals, further wherein each profile may include one or more healthcare services along with a cost;
b) a plurality of comparators, wherein the plurality of comparators include:
i) a service comparator, wherein the service comparator compares the request with the one or more healthcare services at a location identified by the GPS to generate a first output; and
ii) a price comparator, wherein the price comparator compares the price with the cost of the one or more healthcare services to generate a second output;
4) a negotiation module, wherein the negotiation module may be capable of negotiating with one or more healthcare professionals based on the first output and a second output;
further wherein the profiles of the one or more healthcare professionals are displayed on the electronic device based on the negotiation.

2. The platform according to claim 1 may be a software application platform for providing healthcare services to patients.

3. The platform according to claim 1, wherein the healthcare service may be a dental service.

4. The platform according to claim 1, wherein the electronic device may be either of a phone, a laptop, a computer, an iPad and a tablet.

5. The platform according to claim 4, wherein the request from the patient may be either of a voice over, a speech and a text.

6. The platform according to claim 1, wherein the platform provides an interface 118 to the patient for accepting the request for a healthcare service and the price.

7. The platform according to claim 1, wherein the server may be a cloud based server.

8. The platform according to claim 1, wherein the service comparator matches the request with the one or more healthcare services provided by the platform for generating the first output.

9. The platform according to claim 8, wherein the price comparator matches the price with the cost of the one or more healthcare services based on the first output and generates the second output.

10. The platform according to claim 9, wherein the negotiation module negotiates with the one or more healthcare professionals for providing the one or more services matching the request at the price offered by the patient.

11. The platform according to claim 10, wherein the server short-lists a healthcare professional from the one or more healthcare professionals offering the price.

12. The platform according to claim 10, wherein the price may be lower than the cost offered by the one or more healthcare professionals.

13. The platform according to claim 6, wherein the platform provides the patient with a display on the interface 118, wherein the display include a profile of the healthcare professional short-listed by the server.

14. A platform for providing a patient with a healthcare service, the system comprising:
(A) an electronic device for receiving a healthcare request from the patient;
(B) a server, wherein the sever include:
i) a receiver for receiving the healthcare request;
ii) a GPS for locating the electronic device; and
iii) a controller for processing the healthcare request wherein the controller may include:
a) a database, wherein the database maintains profile of healthcare professionals, further wherein each profile may include one or more healthcare services along with a cost;
b) a comparator, wherein the comparator compares the healthcare request with the one or more healthcare services at a location identified by the GPS to generate an output; and
iv) a negotiation module, wherein the negotiation module negotiates the cost between the one or more healthcare professionals based on the output;
further wherein the negotiation module short-lists a healthcare professional from the one or more healthcare professionals based on the negotiation.

15. A method for providing a patient with a healthcare service via a software platform, the method comprising:
receiving a request from an electronic device of the patient on the software platform, wherein the request may include an inquired service from the platform;
processing the request received by the software platform, wherein the processing may include:
identifying location of the electronic device;
accessing information of at least one healthcare professional from a database storing records of a plurality of healthcare professionals, wherein each record may include one or more service provided by each healthcare professional;
comparing the inquired service with the one or more services offered by the plurality of healthcare professionals;
short-listing a number of healthcare professionals based on comparison and the location of the electronic device;
negotiating with the number of healthcare professionals to select one of the number of healthcare professionals; and
displaying record of the one of the number of healthcare professionals on the electronic device to the patient.

16. (canceled)
